(19) United States
(12) Patent Application Publication
(10) Pub. No.: US 2019/0216094 A1
(43) Pub. Date: Jul. 18, 2019

(54) PRODUCT FOR USE AGAINST DEVELOPMENT OR GROWTH OF FUNGUS

(71) Applicant: Fermentationexperts A/S, Bække (DK)

(72) Inventor: Jens Høffner Legarth, Bække (DK)

(21) Appl. No.: 16/312,762

(22) PCT Filed: Jun. 30, 2017
(86) PCT No.: PCT/EP2017/066300

§ 371 Date: Dec. 21, 2018

(30) Foreign Application Priority Data
Jul. 1, 2016 (DK) PA 2016 00393

(51) Int. Cl. A01N 65/08; A01N 65/20; A61K 36/48; A61K 36/31; A23L 33/105; A23K 10/12; A23K 10/30; A23K 10/20; A23L 13/40; A23K 50/30; A23K 50/60

(52) U.S. Cl. CPC A01N 65/08; A01N 65/20; A61K 36/48; A61K 36/31; A23L 33/105; A23V 2002/00; A23K 10/30; A23K 10/20; A23L 13/45; A23K 50/30; A23K 50/60; A23K 10/12

(57) ABSTRACT

The present invention relates to the use of a composition comprising a plant material obtained from the Brassicaceae family and/or the Fabaceae family for inhibiting or reducing growth of a fungus organism.

### TECHNICAL FIELD OF THE INVENTION

The present invention relates to a composition having anti-fungal-activity. In particular the present invention relates to the use of a composition comprising a plant material obtained from the Brassicaceae family and/or the Fabaceae family for reducing or inhibiting growth of a fungus organism.

### BACKGROUND OF THE INVENTION

Unintended microbial growth in different environments is often undesirably as this may lead to spoilage, degradation and/or illness.

Fungus and moulds are a continuing problem in houses and buildings where the humidity is too high and the presence may cause degradation of the infected houses/buildings and result in infecting humans or animals with various kinds of illnesses. It may be difficult to detect moulds in houses/buildings as they may be hidden in the construction but they still represent a serious threat against degradation of the construction and/or illness of human or animals. The pathological challenges with e.g. moulds are that moulds release spores, hyphal fragments, micro-particles and volatile compounds (MVOC) to the air. Presence of mould and spores in houses or buildings may cause humans to feel fatigue, headache, difficulty breathing and other allergy-like symptoms. However, also permanent damages may arise, such as asthma and persistent allergy.

Another challenge with fungal infection, e.g. moulds, is infection and spoilage of food or feed products. In order to stabilise food or feed products, and avoid or postpone spoilage, the food or feed product may be subjected to various preservation treatments, where some has been known since ancient times. The various preservation treatments are provided mainly to inhibit microbial growth, avoid lipid oxidation, or avoid enzymatic activity in the products.

The traditional used preservation methods involve drying, cooling, freezing, boiling, heating, salting, sugaring, smoking, pickling, lye, canning Jellying, jugging, burial, fermentation, pasteurisation, vacuum packing, modified atmosphere, irradiation, and artificial food additives.

The traditionally used preservation methods affect the sensorics appearance (e.g. smell, taste, appearance, consistence etc.), is time consuming or reduce the nutritional benefit of the food or feed product, which is not always desired by the consumer.

Thus, there is a need in the industry for a new method for reducing or limiting the fungal growth and development. In particular there is a need for a new method of and composition for reducing fungal growth, which method is fast, easy, cheap, which maintain or even improve the nutritional value of products and at the same time, limits the effect on the sensorics appearance and thereby avoid some of the disadvantages with the prior art compositions and methods.

### SUMMARY OF THE INVENTION

Thus, an object of the present invention relates to a composition for and a method of reducing or limiting fungal growth and development.

In particular, it is an object of the present invention to provide a composition for and a method of reducing fungal growth, which solves the above problems with the prior art such as undesired sensorics appearance (e.g. smell, taste, appearance, consistence etc.), long handling time or reduced nutritional benefit of the food or feed product.

Thus, one aspect of the invention relates to use of a composition comprising a plant material obtained from the Brassicaceae family and/or the Fabaceae family for inhibiting or reducing growth of a fungus organism.

Another aspect of the present invention relates to an anti-fungal composition comprising a plant material obtained from the Brassicaceae family and/or the Fabaceae family.

Yet another aspect of the present invention relates to a composition for use in alleviation, stabilising or prophylaxis of fungal growth, wherein said composition comprises a plant material, said plant material is obtained from the Brassicaceae family and/or the Fabaceae family.

Still another aspect of the present invention relates to a composition for use in the treatment, alleviation, stabilising or prophylaxis of fungal growth in a mammal (such as a human or an animal), said composition comprises a plant material, said plant material is obtained from the Brassicaceae family and/or the Fabaceae family.

The present invention will now be described in more detail in the following.

### DETAILED DESCRIPTION OF THE INVENTION

Accordingly, unintended development and growth of fungus are often a problem in many areas and there is a continuing interest in reducing, limiting, or avoid unintended growth as this may influence the quality of life or the quality of the food we eat, influence the sensorics experience of food products, cause degradation of the infected houses/buildings and infect of humans or animals with various kinds of illnesses.

Hence, an aspect of the present invention relates to the use of a composition comprising a plant material obtained from the Brassicaceae family and/or the Fabaceae family for inhibiting or reducing growth of a fungus organism.

Additionally, a further aspect of the present invention relates to a method of inhibiting or reducing growth of a fungus organism, by subjecting the fungus organism to a composition comprising a plant material obtained from the Brassicaceae family and/or the Fabaceae family.

Furthermore, an even further aspect of the present invention relates to a composition for use in alleviation, stabilising or prophylaxis of fungal growth, wherein said composition comprises a plant material, said plant material is obtained from the Brassicaceae family and/or the Fabaceae family.

In an embodiment of the present invention the composition further comprises one or more lactic acid bacterial strain.

In a preferred embodiment of the present invention, the composition may be a fermented composition. Preferably, the one or more lactic acid bacterial strain may be responsible for providing the fermented composition.

Thus, a further preferred embodiment of the present invention relates to a fermented composition comprising a plant material obtained from the Brassicaceae family and/or the Fabaceae family and one or more lactic acid bacterial strain, for inhibiting or reducing growth of a fungus organism.

In the present invention, the term “fungus organism” relates to any kind of unwanted or unintended fungal growth or fungal development or growth of fungi in unwanted or unintended places.

In an embodiment of the present invention, the fungus organism is a yeast, a mould and/or a spore hereof. The yeast may preferably be a yeast causing, or capable of causing pathogenic effect in immune-compressed people or cause food spoilage. The mould may preferably be a mould capable of causing food spoilage, property degradation or being pathogenic.

The composition according to the present invention may be the plant material as such (preferably, the plant material may be grinded, cut, chopped, sliced, and/or fractionized), a fraction of the plant material or an extract obtained from said plant material.

A further aspect of the present invention may relate to an anti-fungal composition comprising a plant material obtained from the Brassicaceae family and/or the Fabaceae family.

In the present context, the term “anti-fungal composition” relates to a composition capable of killing, suppressing, reducing, limiting, or avoiding growth or development of fungus organisms. Furthermore, the anti-fungal composition may be capable of killing fungi or fungi spores (acting as a fungicide) or capable of inhibiting fungal growth (acting as a fungistatic)

The anti-fungal composition according to the present invention may be or may comprise the plant material as such, a fraction of the plant material or an extract obtained from said plant material. Preferably, the anti-fungal composition according to the present invention may comprise an extract of the plant material.

In an embodiment of the present invention the composition or the anti-fungal composition may be a disinfectant, a medical product, or an ingredient.

Preferably, the ingredient according to the present invention may form part of a food product, a feed product, a medical product, or a disinfectant.

In the present context, the term “food product” relates to a substance that is suitable for human consumption.

In the present context, the term “feed product” relates to a substance that is suitable for animal consumption.

In an embodiment of the present invention, the food product or the feed product may be a fermented food product or a fermented feed product. Preferably the fermented food product or the fermented feed product comprise, in addition to the plant material, one or more lactic acid bacterial strain.

The food product or the feed product may comprise in the range of 0.1-15% (w/w) on a dry matter basis of the plant material, selected from the Brassicaceae family, the Fabaceae family or a combination thereof, such as in the range of 0.5-13%, e.g. in the range of 1-12%, such as in the range of 2-11%, e.g. in the range of 3-10%, such as in the range of 5-9%, e.g. in the range of 6-8%.

In a further embodiment of the present invention the fermented food product or the fermented feed product may be a meat product or a meat comprising product.

In yet an embodiment of the fermented food product, the meat product or the meat comprising product may be a cured product, such as a sausage.

In the present context, the term “medical product” relates to a substance that is suitable for treatment, alleviation, stabilising or prophylaxis of fungal growth in a mammal, such as a human or an animal.

Hence, an embodiment of the present invention relates to a composition for use in the treatment, alleviation, stabilising or prophylaxis of fungal growth in a mammal, such as a human or an animal, said composition comprises a plant material, said plant material may be obtained from the Brassicaceae family and/or the Fabaceae family.

In a further embodiment of the present invention, the medical product may be in the form of oil, cream or an ointment.

In the present context, the term “disinfectant” relates to a substance that is suitable for destroying, neutralizing, or inhibiting the growth of fungi, in particular disease-carrying fungi.

One advantage of the disinfectant of the present invention is that it is a natural product which has very low environmental pressure and no harmful chemicals or components, harmful to the environment, are emitted to the environment.

In an embodiment of the present invention the fermented composition or the fermented anti-fungal composition comprises a glucosinolate content of at least 2 μmol/g on a dry matter basis. In a further embodiment of the present invention the fermented composition or the fermented anti-fungal composition may have a glucosinolate content of at least 3 μmol/g on a dry matter basis, such as at least 4 μmol/g on a dry matter basis, e.g. at least 5 μmol/g on a dry matter basis, such as at least 7.5 μmol/g on a dry matter basis, e.g. at least 10 μmol/g on a dry matter basis, such as at least 12.5 μmol/g on a dry matter basis, e.g. at least 15 μmol/g on a dry matter basis, such as at least 17.5 μmol/g on a dry matter basis, e.g. at least 20 μmol/g on a dry matter basis, such as at least 25 μmol/g on a dry matter basis, e.g. at least 30 μmol/g on a dry matter basis, such as at least 40 μmol/g on a dry matter basis, e.g. at least 50 μmol/g on a dry matter basis, such as at least 60 μmol/g on a dry matter basis, e.g. at least 70 μmol/g on a dry matter basis, such as at least 80 μmol/g on a dry matter basis, e.g. at least 90 μmol/g on a dry matter basis, such as at least 100 μmol/g on a dry matter basis.

In an embodiment of the present invention the content of glucosinolates present in the fermented composition or the fermented anti-fungal composition may be at least 10% (w/w) of the glucosinolates naturally present in the plant material, such as at least 10% (w/w), e.g. at least 20% (w/w), such as at least 30% (w/w), e.g. at least 40% (w/w), such as at least 50% (w/w), e.g. at least 60% (w/w), such as at least 65% (w/w), e.g. at least 70% (w/w), such as at least 75% (w/w), e.g. at least 80% (w/w), such as in the range of 10-95% (w/w) of the glucosinolates naturally present in the plant material, e.g. in the range of 15-75% (w/w), such as in the range of 20-50% (w/w), e.g. in the range of 30-85% (w/w), such as in the range of 50-75% (w/w).

In the present context, the term “glucosinolates naturally present in the plant material” relates to a determined/analysed content of glucosinolates in the plant material to be fermented—before fermentation. If no determination or analysis of the plant material before fermentation has been made or can be made, the “glucosinolates naturally present in the plant material” relates to the amount of glucosinolates to be found in the literature for the specific plant material.

The Plant Material

The plant material according to the present invention is obtained from the Brassicaceae family, the Fabaceae family or a combination of the Brassicaceae family and the Fabaceae family.

Preferably, the Brassicaceae family is selected from Brassica spp., cruciferous vegetables, cabbage species, and/or mustard species.

In an embodiment of the present invention, the Brassica spp. may preferably be selected from one or more of rape species, a broccoli species and/or a cauliflower species. Preferably, the rape species is a rapeseed product, such as rapeseed meal, or rapeseed cake, preferably rapeseed cake.

In another embodiment of the present invention, the Brassica spp. may be selected from one or more species such as Brassica napus; Brassica oleracea; Brassica campestris; and/or Brassica rapa.

In an embodiment of the present invention, the plant material comprises a combination of 2 or more plant material species obtained from the Brassicaceae family, such as 3 or more, e.g. 4 or more, such as 5 or more, e.g. 6 or more.

Preferably, Fabaceae family may be selected from the Faboideae subfamily. Even more preferably, the Fabaceae family and/or the Faboideae subfamily may be selected from the Glycine genus. Even more preferably, the Fabaceae family is selected from G. Max species. Even more preferably, the Fabaceae family may be selected from soya plant.

In a preferred embodiment of the present invention, the plant material does not comprise a seaweed material or an algae material.

In an embodiment of the present invention the plant material does not comprise other plant materials, then those obtained from the Brassicaceae family.

In an embodiment of the present invention the plant material does not comprise other plant materials, then those obtained from the Fabaceae family.

In a further embodiment of the present invention, the plant material comprises a combination of a plant material obtained from the Brassicaceae family and a plant material obtained from the obtained from the Fabaceae family. Preferably, the plant material does not comprise other plant materials than the plant material obtained from the Brassicaceae family and the plant material obtained from the obtained from the Fabaceae family. Preferably, the plant material comprises at least 50% (w/w) dry-matter plant material obtained from the Brassicaceae family, such as at least 65% (w/w), e.g. at least 75% (w/w).

The one or more lactic acid bacterial strain

In a further embodiment of the present invention the one or more lactic acid bacterial strain present in the composition or in the fermented composition may be selected from the group consisting of Lactobacillus, Leuconostoc, Pediococcus, Lactococcus, Streptococcus, Aerococcus, Carnobacterium, Enterococcus, Oenococcus, Teragenococcus, Vagococcus, and Weisella. Preferably, the one or more lactic acid bacterial strain present in the composition or in the fermented composition are preferably lactic acid bacteria of the genus Enterococcus, Lactobacillus, Pediococcus, Lactococcus, or a combination thereof.

In an even further embodiment of the present invention the one or more lactic acid bacteria stain may be selected from the group consisting of one or more Enterococcus spp., Lactobacillus spp., Lactococcus spp., Pediococcus spp., and a combination hereof. Preferably, the one or more lactic acid bacterial strain is selected from the group consisting of one or more of Enterococcus faecium, Lactobacillus rhamnosus, Lactobacillus plantarum, Pediococcus acidililactili, Pediococcus pentosaceus, Lactococcus Lactis, Lactococcus Cremoris, Lactococcus Diacetylactis, Leuconostoc Cremoris and a combination hereof.

In yet a further embodiment of the present invention, the main lactic acid bacterial strain present in the composition or in the fermented composition may be Pediococcus pentosaceus; Pendiococcus acidilactici; Lactobacillus plantarum; Lactobacillus rhamnosus; or Enterococcus faecium. Preferably, the main lactic acid bacteria present in the composition may be Lactobacillus plantarum.

In another embodiment of the present invention the one or more lactic acid bacteria stain(s) may be selected from the group consisting of one or more of Enterococcus faecium MCIMB 30122, Lactobacillus rhamnosus NCIMB 30121, Pediococcus pentosaceus HTS (LMG P-22549), Pendiococcus acidilactici NCIMB 30086 and/or Lactobacillus plantarum LSI (NCIMB 30083).

In order to provide the desired effects, the fermented composition should have a high content of viable lactic acid bacteria. In an embodiment of the present invention the fermented composition comprises one or more lactic acid bacterial strain in a total amount in the range of 10⁵-10¹² CFU per gram, such as in the range of 10⁸-10¹² CFU per gram, e.g. in the range of 10⁷-10¹¹ CFU per gram, such as in the range of 10⁸-10¹¹ CFU per gram, e.g. in the range of 10⁹-10¹⁰ CFU per gram.

In an embodiment of the present invention the one or more lactic acid bacterial strain may be added to the plant material in a concentration sufficient to outgrow any microorganisms, such as bacteria and/or fungus originally present in the plant material.

In order to improve the fermentation process and the effect of the fermented composition the fermented composition may comprise fractionized plant material. The plant material may be fractionized by grinding, cutting, chopping, slicing, and/or fractionizing providing a fractionized plant material.

The Method for Providing the Composition

One aspect of the present invention relates to a process for preparing a fermented composition according to the present invention, in particular a fermented anti-fungal composition comprising a plant material (having prebiotic activity) and one or more lactic acid bacterial strain (having probiotic activity), the method comprises the steps of:
(i) providing a plant material;
(ii) providing one or more lactic acid bacteria strain;
(iii) subjecting the plant material to fermentation by combining the plant material provided in step a) with the one or more lactic acid bacterial strain provided in step b),
(iv) continuing the fermentation until the fermented composition is provided having a pH value below 5.0

In an embodiment of the present invention one or more probiotic bacterial may be lactic acid-producing bacteria, lactic acid bacteria.

Lactic acid bacteria may produce lactic acid and other metabolic products which contribute to the organoleptic, textural, nutritional and pharmacological profile of the fermented composition.

Thus, depending on how and where the composition of the present invention is to be used, and depending on the concentration of the composition to be used, the fermentation process may be controlled to limit or negeleidge the influence of the composition on the final product, in particular in respect of one or more of the organoleptic, the textural, the nutritional or the pharmaceutical properties on the final product.

The industrial importance of the lactic acid bacteria may be evidenced by their generally regarded as safe (GRAS) status, due to their ubiquitous appearance in food and their contribution to the healthy microflora of human mucosal surfaces. The genera that comprise the lactic acid bacteria, and which may be used in the present invention, are Lactobacillus, Leuconostoc, Pediococcus, Lactococcus, and Streptococcus, Aerococcus, Carnobacterium, Enterococcus, Oenococcus, Teragenococcus, Vagococcus, and Weisella; these genera belong to the order Lactobacillales.

In order to increase productivity and effectivity two or more lactic acid bacterial strains may be provided, such as three or more lactic acid bacterial strains, e.g. four or more lactic acid bacterial strains, such as 7 or more lactic acid bacterial strains, e.g. 10 or more lactic acid bacterial strains, such as 15 or more lactic acid bacterial strains, e.g. 20 or more lactic acid bacterial strains, such as 25 or more lactic acid bacterial strains, e.g. 30 or more lactic acid bacterial strains, such as 35 or more lactic acid bacterial strains, e.g. 40 or more lactic acid bacterial strains.

In an embodiment of the present invention a starter culture or an inoculum may be provided comprising one or more lactic acid bacterial strain as defined herein.

The term “inoculum” relates to a source material, such as the one or more lactic acid bacterial strain, used for the inoculation of a new culture. The inoculum may be employed to prime a process of interest.

“Inoculation” refers to the placement of a microorganism (e.g. one or more lactic acid bacterial strain) that will grow when implanted in a culture medium such as a fermentation tank comprising media to be fermented, e.g. a plant material.

A primary inoculum may be provided and refers to the generation of an initial inoculum in a series of repeated similar of essentially identical inoculation process, for example one or more repetitions of a fermentation process. An aliquot of the product of the formation process may be used to inoculate a new process of fermentation. Thus, the inoculation may be a fermented feed product which comprises viable lactic acid producing bacteria in sufficient amount to prime a lactic acid fermentation process of a another feed product, or similar feed, to be fermented, e.g. a plant material.

The inoculum according to the present invention may be a in a liquid form, dry form, or essentially dry form. The moisture content of the inoculum may be adjusted in order to optimize the fermentation process. In an embodiment of the present invention, the inoculum may be provided as essentially pure viable bacteria (such as bacteria in freeze dried form) or bacteria suspended in a suitable media prior to the application (such as a water, buffer or a growth media).

The proportion of the inoculums added to the plant material may vary. In case it is considered that the load of undesirable microbes is significant in the plant material or the fermentation system, the proportion of the inoculum in the fermentation mixture (inoculum+plant material+additional water) may be increased to ensure that the fermentation is directed by the microbes (e.g. lactic acid bacteria) of the inoculums. Thus, the inoculum may be provided with a concentration of lactic acid bacteria in the inoculum sufficient to outgrow other (non-lactic acid bacteria, yeast or moulds present in the plant material.

Accordingly, in one embodiment of the invention, the proportion of the inoculums in the combination of the plant material and the one or more lactic acid material as defined in step (iii) is in the range of 0.1 to 99.9 vol-%; such as 1 to 99 vol-%; e.g. 5 to 70 vol-%; such as 10 to 50 vol-%; e.g. 25 to 35 vol-%; such as 0.1 to 10 vol-%; e.g. 0.5 to 5 vol-15%; such as 1 to 2.5 vol-%; or around 1 to 2 vol-%.

The fermentation process according to the present invention, may preferably be essentially a homofermentative process. “Essentially homofermentative” means, that the predominant bacterial flora driving the fermentation is homofermentative. In the present context, the term “essentially homofermentative” relates to a fermentation process where, 99% or more of the bacteria are homofermentative, such as 95% or more of the bacteria are homofermentative, e.g. 90% or more of the bacteria are homofermentative, such as 85% or more of the bacteria are homofermentative, e.g. 80% or more of the bacteria are homofermentative, such as 70% or more of the bacteria are homofermentative, e.g. 60% or more of the bacteria are homofermentative.

In an embodiment of the present invention the fermentation is essentially a homofermentation, such as a homolactic fermentation.

The term “homolactic fermentation” when used according to the present invention indicates that the major fermentation product is lactic acid, and the levels of acetic acid and ethanol are either below taste threshold, around taste threshold or slightly above taste threshold. Preferably, “essentially homofermentative” indicates a ratio of lactic acid to acetic acid or lactic acid to ethanol in (mM/mM) of more than 1:1, such as 2:1 or more, e.g. 10:1 or more, such as 20:1 or more, e.g. 50:1 or more, or such as 100:1 or more.

In another embodiment of the present invention the method according to the present invention is essentially a heterofermentative fermentation.

In the present context, the term “essentially heterofermentative” means, that the predominant bacterial flora driving the fermentation is heterofermentative. In the present context the term “essentially heterofermentative” relates to a fermentation process where, 99% or more of the bacteria are heterofermentative, such as 95% or more of the bacteria are heterofermentative, e.g. 90% or more of the bacteria are heterofermentative, such as 85% or more of the bacteria are heterofermentative, e.g. 80% or more of the bacteria are heterofermentative, such as 70% or more of the bacteria are heterofermentative, e.g. 60% or more of the bacteria are heterofermentative.

In an embodiment of the present invention the heterofermentation according to the present invention results in a major part of the fermentation products are acetic acid and ethanol, and the ratio of acetic acid and ethanol to lactic acid in (mM/mM) is more than 1:1, such as 2:1 or more, e.g. 10:1 or more, such as 20:1 or more, e.g. 50:1 or more, or such as 100:1 or more.

In a further embodiment of the present invention, the fermented composition provided in step iv) has an acetic acid and/or ethanol concentration of at least 50 mM, such as at least 100 mM, such as 100-1000 mM, such as 100-500 mM, such as 100-300 mM, such as 100-200 mM, such as 150-500 mM, such as 200-500 mM or such as 300-500, mM lactic acid.

The fermentation according to the present invention may be continued for 10 days or less, such as 9 days or less, e.g. 8 days or less, such as 7 days or less, e.g. 6 days or less, such as 5 days or less, e.g. 4 days or less, such as 3 days or less, e.g. 2 days or less, such as 36 hours or less, e.g. 24 hours or less.

Even the fermentation should be continued as quickly as possible, the inventors also found that a certain time of fermentation may be required to provide the desired nutritional and/or pharmacological effects of the fermented composition. Hence, in an embodiment of the present invention the fermentation should be continued for more than 2 hours, such as more than 5 hours, e.g. more than 8 hours, such as more than 10 hours, e.g. more than 12 hours, such as more than 15 hours, e.g. more than 17 hours, such as more than 20 hours, e.g. more than 24 hours, such as in the range of 24 hours to 10 days, e.g. in the range of 20 hours to 8 days, such as in the range of 15 hours to 6 days, e.g. in the range of 12 hours to 4 days, such as in the range of 10 hours to 2 days, e.g. in the range of 8 hours to 36 hours, such as in the range of 8 hours to 24 hours, e.g. in the range of 2 hours to 20 hours, such as in the range of 6 hours to 15 hours.

Controlling the temperature may be one of the best ways to improve the quality and the effects of the fermented composition. In an embodiment of the present invention, the fermentation is performed at a temperature below 50° C., such as below 47° C., e.g. below 45° C., such as below 43° C., e.g. in the range 15-45° C., such as 18-43° C., such as 25-40° C., such as 30-40° C., such as 15-20° C. or such as 40-45° C.

The fermentation process may preferably involve a temperature gradient, said temperature gradient involves 3 stages; a starting temperature, a temperature increase and a steady state fermentation temperature.

In an embodiment of the present invention the starting temperature may be in the range of 18-30° C., such as in the range of 20−28° C., e.g. in the range of 22-26° C., such as in the range of 24-25° C.

The temperature increase of the fermentation process of the present invention may preferably be a slow temperature increase from the starting temperature to the steady state fermentation temperature. In an embodiment of the present invention the temperature increase is provided without addition of heat. In the present context, the term “without addition of heat” relates to a fermentation temperature increase wherein the heat provided is produced by the fermentation itself without the use of electrical, mechanical or fuel based heat.

In an embodiment of the present invention, the steady state fermentation temperature may be below 50° C., such as below 47° C., e.g. below 45° C., such as below 43° C., e.g. in the range 20-45° C., such as in the range 22-43° C., such as in the range 25-40° C., such as in the range 30-40° C., such as in the range 40-45° C.

In yet an embodiment of the present invention, the fermentation may be a one-step fermentation of the plant material.

In the present context, the term “one-step fermentation” relates to a fermentation process wherein the same type of plant material is subjected to the same fermentation conditions, or substantially the same fermentation conditions. Hence, the term “one-step fermentation”, exclude the option of taking out a part of the plant material during fermentation, leaving the remaining plant material to be further fermented and followed by mixing the part which was taken out with the further fermented material.

In a further embodiment of the present invention, the fermented composition does not involve subsequent supplementation of plant material and/or one or more lactic acid bacterial strain(s) to the fermented composition.

The moisture content of the plant material to be fermented may be another relevant parameter to control in order to control the fermentation process and the resulting fermented composition. Thus, in an embodiment of the present invention the moisture content during the fermentation may be in the range of 20-60% (w/w), such as in the range of 30-50% (w/w), e.g. preferably in the range of 35-45% (w/w).

In a preferred embodiment of the present invention, the fermentation may be continued until the fermented composition has a pH below pH 6.5, such as below pH 6.0, e.g. below pH 5.5, such as below pH 5.0, e.g. in the range of pH 3.0-6.5, such as in the range of pH 3.0-6.0, e.g. in the range of pH 3.1-5.5, such as in the range of pH 5.0-3.2, such as in the range 3.3-4-2, such as 3.4-4.0, such as 3.5-3.8, such as 3.7-4.2, such as 3.7-4.0, or such as 3.8-4.2.

In yet another embodiment, the fermented composition provided in step iv) has a lactic acid concentration of at least 50 mM, such as at least 100 mM, such as at least 250 mM, such as at least 500 mM, such as at least 750 mM, such as at least 1000 mM, such as 100-1000 mM, such as 100-500 mM, such as 100-300 mM, such as 100-200 mM, such as 150-500 mM, such as 200-500 mM or such as 300-500, mM lactic acid.

Since it is believed that the probiotic effect of the one or more lactic acid bacterial strain peaks after 2 hours to 20 days, such as 3 hours to 15 days, e.g. 6 hours to 10 days, such as 12 hours to 9 days, e.g. 18 hours to 8 days, such as 24 hours to 7 days, e.g. 2-6 days, such as after 3-5 days, e.g. after 4-5 days, such as 6 hours to 4 days, such as 6 hours to 3 days, e.g. 6 hours to 2 days, such as 6 hours to 24 hours, e.g. 6 hours to 18 hours, such as 6 hours to 12 hours the fermentation process may be stopped within this time period. After the fermentation is stopped the fermented composition may be dried in order to prolong the viability (CFU) of the one or more lactic acid bacterial strain.

The fermented composition according the present invention may be a liquid, a slurry, or a dry powder.

The process according to the present invention may further comprises a step of reducing the moisture content from the moisture content obtained from the fermentation process to a fermented composition moisture content. In an embodiment of the present invention the composition may be subjected to drying.

Any moisture reducing methods may be used which are sensitive to the pre- and probiotic components of the fermented composition in order to maintain the activity of the components in the fermented composition and that ensure high viability of the lactic acid bacteria present in the fermented composition. The method to reduce to moisture content as described in WO 2013/029632 may be preferred (WO 2013/029632 is hereby incorporated by reference).

In an embodiment of the present invention, the moisture content of the composition according to the present invention may be at most 10% (w/w), such as at most 8% (w/w), e.g. at most 6% (w/w), such as at most 4% (w/w), e.g. at most 2% (w/w).

In order to improve the fermentation process, the plant material, according to the present invention, may be pre-treated before combining the plant material with the one or more lactic acid bacterial strain as described in step (iii).

Such pre-treatment may involve grinding, cutting, chopping, slicing, and/or fractionizing the plant material.

In an embodiment of the present invention the plant material, in particular Brassica napus; or Brassica campestris, may be subjected to a pre-treatment to an extent that result in an average diameter which is at most 75% of the average diameter of the plant material, such as at most 50%, e.g. at most 25%, such as at most 10% of the average diameter.

In a further embodiment of the present invention the plant material may be subjected to a pre-treatment providing a pre-treated plant material having an average diameter of 3 mm or less, such as an average diameter of 2 mm or less, such as an average diameter of 1 mm or less, such as an average diameter in the range 25 μm to 3 mm, such as 0.1 mm to 2.5 mm, such as an average diameter in the range of 0.5 mm to 2.25 mm, such as an average diameter in the range 1.0 mm to 2 mm.

It should be noted that embodiments and features described in the context of one of the aspects of the present invention also apply to the other aspects of the invention.

The invention will now be described in further details in the following non-limiting examples.

### EXAMPLES

### Example 1

The aim of the present example was to analyse the health effect of dried, fermented rapeseed and/or soya supplemented meals on fungus development in pregnant, lactating sows and piglets.

The Sows and Piglets Tested

The example was conducted on a pig farm in Lipowiec, Lubuskie Province, Poland. Research on the usage of fermented rapeseed and/or soya meal was divided into two phases. The first phase was conducted on 40 sows of the Polish Large White and Polish Landrace breed during their pregnancy and lactating period, whereas the second phase on 180 post-weaning piglets (28 days), derived from the sows used for Phase I of the example. Research included sows after their second and third lactation. Laboratory analysis was performed in the Department of Biochemistry and Toxicology as well as the Institute of Animal Feeding and Bromatology UP in Lublin. The second phase on a hundred and eighty post-weaning piglets, derived from the sows used for Phase I of the example, starting from the twenty-ninth day of the piglets' life.

Design of the Example

In the first phase of the experiment, sows (after confirmed pregnancy) were divided equally into two groups created on a basis of analogue by body weight and their next reproductive cycle (Table 1). Animals from the C_{L} group, were fed with regular type PR-premixes (pregnancy premixes) during their pregnancy and LA-premixes (lactating premixes) during the lactating period. Nutrient contents were in line with the Feeding guidelines and food value for pigs (2014). Animals from the FR_{L} group were fed with the same premixes as Group I (the C_{L} group), but with a 4% addition of fermented rapeseed in the meal (Table 1).

TABLE 1
Design of the experiment Phase I and II

Feeding groups

| Specification | C_{L} | FR_{L} |
|---|---|---|
| Experimental factor (%) | | |
| fermented rapeseed (%) | 0 | 4 |
| Animal count | 10 | 30 |

| Phase II | CO | C | FR | FR/FS | FS/FR | FS |
|---|---|---|---|---|---|---|
| Phase II of the example | | | | | | |
| piglets | S_{CO} | S_{C} | S_{FR} | S_{FR/FS} | S_{FS/FR} | S_{FS} |
| Experimental factor (%) | | | | | | |
| fermented rapeseed (%) | 0 | 0 | 8 | 6 | 2 | 0 |
| fermented soya meal (%) | 0 | 0 | 0 | 2 | 6 | 8 |
| Animal Count | 30 | 30 | 30 | 30 | 30 | 30 |
| Phase II duration | 80 | 80 | 80 | 80 | 80 | 80 |
| (day of the life) | | | | | | |
| Piglets weaning date, days | 28 | 28 | 28 | 28 | 28 | 28 |

C_{L}—control sows;
FR_{L}—sows receiving the experimental mix
CO—piglets control group (derived from sows not receiving the experimental mix)
C—piglets control group (derived from sows receiving the experimental mix)
S—Starter premix for adequate experimental group
FR—piglets receiving the experimental mix comprising fermented rape
FS—piglets receiving the experimental mix comprising fermented soya

Phase II of the example was conducted on piglets derived from Phase I sows. This phase started the day after the piglets delivered by the sows and ended once the piglets reached 30 kg of body weight.

30 piglets each from both sow groups—C_{L} and FR_{L}—made up two respective control groups—control CO and C. The piglets from control group CO and C received the Starter premix (without the experimental factor), in which the nutrient contents were in line with the Feeding guidelines and food value for pigs (2014). The four groups (FR; FR/FS; FS/FR and FS) were created from 120 piglets derived from sows of the FR_{L} group. The piglets from these four groups received the same Starter premix as the control group piglets, but with the 8% addition of the experimental components (either fermented rape, fermented soya or two different combinations hereof). The FR group received a premix with an 8% addition of fermented rapeseed supplement, the FR/FS group a premix with a 6% addition of fermented rapeseed supplement and a 2% addition of fermented soya meal supplement, the FS/FR group received a premix with a 2% addition of fermented rapeseed supplement and a 6% addition of fermented soya meal supplement, and the FS group was fed with a premix containing 8% of fermented soya meal supplement.

Sows during their pregnancy were held in coops of five, however during their labor and lactating period they were held separately. The dry premix (or the premix comprising the fermented composition) was moisturized in a ratio of 1:2.5. Serving sizes were weighted for two sows in a cage. Sows during their pregnancy and lactating periods had a free access to soothie pacifier. Premixes for piglets and porkers were inflicted a libitum, with permanent access to drinking water. Conditions in the pig farms were in line with the recommended zoohigienic norms described by Rokicki, Kolbuszewski 1996, and the animals were under a constant medical care of a veterinarian.

Microbiological Analysis

Material for microbiological analysis was made up of small bowel's contents as well as piglets' faeces. Faeces were analyzed towards the end of the experiment, and contents of the small bowel were collected from chosen, slaughtered individuals from each group at the end of the experiment. Bowel and faeces samples were collected into sterile containers and cooled down to 4° C. Material was homogenized in order to create a batch group, separate for each of the groups in the example. 20 g of prepared material was weighted and put in sterile bottles containing 180 ml of Ringer's solution. This solution was vortexed for five minutes and then left to sediment for fifteen minutes. Serial dilutions were composed from the obtained solution and then seeded into a Petri dish with adequate microbiological substrate.

Next, the dishes were incubated and fungus and mold was allowed to growth on DG18 substrate, for 5-7 days at temperature of 25° C.

After the specific incubation time, the grown colonies were counted with an automated counter and converted accordingly to the PN-ISO 4832, PN-EN ISO 7218 norms.

In order to identify yeast-like fungi, grown colonies were evaluated macroscopingly and then sifted reductively onto a Sabourauda substrate. The colonies were evaluated macro- and microscopingly and then Gram-stained. Final identification was made based on the available tests API2OCAUX (bioMérieux Poland).

All tests were performed in line with PN-ISO 4832, PN-EN ISO 7218.

Statistical Analysis

Obtained figures were put under a statistical analysis, using a Statistica version 5 program, single factor analysis of variance ANOVA taking the materiality level of 0.05 and 0.01

Results:

The results of the analysis of dried, fermented rapeseed and/or soya supplemented meals on fungus development in pregnant, lactating sows and piglets are summarized in the below Table 2 and Table 3 and is illustrated in the enclosed FIG. 1 and FIG. 2.

TABLE 2
Fungal development in piglets small intestines

| | Experimental group | | | | | |
|---|---|---|---|---|---|---|
| | CO | C | FR | FR/FS | FS/FR | FS |
| | GENERAL FUNGUS COUNT | | | | | |
| CFU/gr | 7309 | 3700 | 1350 | 3468 | 4095 | 3255 |
| decimal notation | 7.3 × 10³ | 3.7 × 10³ | 1.3 × 10³ | 3.5 × 10³ | 3.6 × 10³ | 3.2 × 10³ |
| log 10 | 3.9 | 3.6 | 3.1 | 3.5 | 3.6 | 3.5 |

TABLE 3
Fungal development in piglets faeces'

| | Experimental group | | | | | |
|---|---|---|---|---|---|---|
| | CO | C | FR | FR/FS | FS/FR | FS |
| | GENERAL FUNGUS COUNT | | | | | |
| CFU/gr | 320 | ND | 50 | 41 | 105 | 14 |
| decimal | 3.2 × 10² | ND | 5 × 10¹ | 4.1 × 10¹ | 1.0 × 10² | 1.4 × 10¹ |
| notation | | | | | | |
| log 10 | 2.5 | ND | 1.7 | 1.6 | 2.0 | 1.1 |

In the above numbers obtained from table 2 and table 3 (and as shown in FIG. 1 and FIG. 2) a clear and significant decrease in fungus development are demonstrated in both the intestines and in the faeces of the piglets. Fermented rape alone shows to have the strongest effect against the fungus development in the intestine, whereas the fermented soya or the combination of fermented soya and fermented rape show to have the strongest effect on the fungal count of the faeces.

1. A method of using a fermented composition comprising a plant material obtained from the Brassicaceae family and/or the Fabaceae family for inhibiting or reducing growth of a fungus organism.

2. The method according to claim 1, wherein the plant material is subjected to a pre-treatment to an extent that result in an average diameter which is at most 75% of the average diameter of the plant material, such as at most 50% of the average diameter.

3. The method according to claim 1, wherein the plant material is subjected to a pre-treatment providing a pre-treated plant material having an average diameter of 3 mm or less, such as an average diameter of 2 mm or less, such as an average diameter of 1 mm or less, such as an average diameter in the range 25 μm to 3 mm, such as 0.1 mm to 2.5 mm, such as an average diameter in the range of 0.5 mm to 2.25 mm, such as an average diameter in the range 1.0 mm to 2 mm.

4. The method according to claim 1, wherein the fungus organism is a yeast or a mould and/or a spore hereof.

5. The method according to claim 1, wherein the plant material is subjected to a fermentation process.

6. The method according to claim 5, wherein the fermentation process involves one or more lactic acid bacterial strain(s).

7. The method according to claim 6, wherein the one or more lactic acid bacterial strain(s) is selected from the group consisting of Lactobacillus, Leuconostoc, Pediococcus, Lactococcus, Streptococcus, Aerococcus, Carnobacterium, Enterococcus, Oenococcus, Teragenococcus, Vagococcus, and Weisella.

8. The method according to claim 1, wherein the composition comprises a high content of viable lactic acid bacteria.

9. The method according to claim 1, wherein the composition is an ingredient and wherein the ingredient is part of a food product, a feed product, a medical product, or a disinfectant.

10. The method according to claim 9, wherein the food product or the feed product is a fermented food product or a fermented feed product.

11. The method according to claim 10, wherein the fermented food product or the fermented feed product is a meat product or a meat comprising product.

12. An anti-fungal composition comprising a fermented plant material obtained from the Brassicaceae family and/or the Fabaceae family.

13. The anti-fungal composition according to claim 12, wherein the composition comprises an extract of the plant material.

14. The anti-fungal composition according to claim 12, wherein the antifungal composition is an ingredient, and wherein the ingredient is an ingredient for a food product or a feed product.

15. A composition for use in alleviation, stabilizing or prophylaxis of fungal growth, wherein said composition comprises a fermented plant material, said plant material is obtained from the Brassicaceae family and/or the Fabaceae family.

16. A composition for use in the treatment, alleviation, stabilising or prophylaxis of fungal growth in a mammal (such as a human or an animal), said composition comprises a fermented plant material, said plant material is obtained from the Brassicaceae family and/or the Fabaceae family.

17. (canceled)
